(19) United States
(12) Patent Application Publication
(10) Pub. No.: US 2022/0347206 A1
(43) Pub. Date: Nov. 3, 2022

(54) METHODS OF PROPHYLAXIS AND TREATMENT OF COVID-19 USING AZOXIMER BROMIDE

(71) Applicant: NPO Petrovax Pharm, LLC, Moscow (RU)

(72) Inventors: Sergey V. Efimov, Moscow (RU); Liaysan R. Mukhamadieva, Moscow (RU); Vera P. Vavilova, Moscow (RU); Nadezhda F. Khomyakova, Moscow (RU); Yulia S. Karpova, Moscow (RU)

(21) Appl. No.: 17/246,400

(22) Filed: Apr. 30, 2021

(51) Int. Cl. A61K 31/787; A61K 45/06; A61K 31/4706; A61P 31/14

(52) U.S. Cl. CPC A61K 31/787; A61K 45/06; A61K 31/4706; A61P 31/14

(57) ABSTRACT

Methods for treatment of people afflicted with COVID-19 are described. The methods include administration of azoximer bromide to people afflicted with COVID-19. Methods of prophylaxis against COVID-19 by administering azoximer bromide are also described herein. The methods include administration of azoximer bromide to healthy people not afflicted with COVID-19. Azoximer bromide may be administered intravenously, as a pill, nasal spray, and/or suppository, with each treatment dose being designed to complement the mode of administration. Azoximer bromide may be administered for treating COVID-19 as part of a complex therapy including at least one of a vitamin, mineral, antibiotic, antiviral, immunosuppressant, hydroxychloroquine, and anti-coagulant.

### FIELD

This disclosure generally relates to prophylaxis and treatment for viral infections, and, more particularly to prophylaxis for preventing COVID-19 infections and to treatments of COVID-19 infections.

### BACKGROUND

The clinical presentation of coronavirus disease 2019 (COVID)-19 ranges from asymptomatic infection to fatal illness, and is considered to be caused by the severe acute respiratory syndrome coronavirus 2 virus (SARS-CoV-2). SARS-CoV-2 primarily targets the respiratory system and can cause pneumonia and respiratory failure. High infection rates of people with COVID-19 are generally associated with high rates of intensive care admission. Worldwide, about 150 million cases of COVID-19 infections have been reported, and over 3 million deaths from COVID-19 have been reported.

While about 200 candidate vaccines against COVID-19 are known to be in various stages of development, evidence-based therapies are currently limited. Known COVID-19 treatments include remdesivir, a broad-spectrum antiviral shown to modestly reduce time to recovery in hospitalized adults and dexamethasone, an anti-inflammatory shown to reduce mortality in patients requiring respiratory support. Antiviral antibody cocktails have also demonstrated some capacity to reduce the viral load of COVID-19 in non-hospitalized patients, while combination therapy with monoclonal antibodies bamlanivimab and etesevimab has been recently shown to reduce viral load in outpatients with mild-to-moderate disease. Currently, there is a recognized need and urgency to find viable treatments for COVID-19.

Notably, healthcare workers around the world are paying a very high price in the fight against COVID-19. Thousands of doctors have contracted the virus, and the number of doctors seriously ill as a result of being infected with COVID-19 is steadily growing. The World Health Organization (WHO) has commented that healthcare workers are disproportionately affected by the COVID-19 pandemic, since they make up only 3% of the population, but account for 14% of all reported cases of COVID-19 infection.

Health workers who come into contact with people with coronavirus infection or those who are potential illnesses are at increased risk of infection. For this reason, various state authorities are seriously concerned about the protection of medical personnel against COVID-19. To enhance protection, specialists are constantly working on the development and improvement of preventive/prophylactic measures. At the present time, there are already basic safety rules designed to prevent COVID-19 infection of at-risk health care professionals. However, the medical staff working in close proximity to individuals infected with COVID-19 are not only susceptible to infection, but they are risking exposure to a high viral load. Thus, such medical personnel not only has a higher chance of getting sick compared to others, but their disease can be more severe.

### BRIEF DESCRIPTION OF THE DRAWINGS

Disclosed herein are embodiments of systems and methods pertaining to treatment of COVID-19 and associated symptoms, as well as methods of prophylaxis against COVID-19. This description includes drawings, wherein:

FIG. 1 is a graph that depicts the Evolution of Ordinal Scale score and time-to-event analysis during the COVID-19 treatment and follow-up phases described in Example 1, presented overall, and shows the Mean (standard deviation or SD) Ordinal Scale Scores during the treatment phase;

FIG. 2 is another graph that depicts the Evolution of Ordinal Scale score and time-to-event analysis during the treatment and follow-up phases described in Example 1, presented overall, and shows the Mean Ordinal Scale Scores during the treatment phase stratified by the baseline NEWS severity: ‘Very Severe’ (score≥9); ‘Severe or Moderate’ (score 5-8), and ‘Mild’ (score≤4);

FIG. 3 is another graph that depicts the Evolution of Ordinal Scale score and time-to-event analysis during the treatment and follow-up phases described in Example 1, presented overall, and shows the Mean Ordinal Scale Scores during the treatment phase stratified by patient age: <65 years and ≥65 years;

FIG. 4 is another graph that depicts the Evolution of Ordinal Scale score and time-to-event analysis during the treatment and follow-up phases described in Example 1, presented overall, and shows the improvement probability curve plotted for time to decreasing Ordinal Scale score to ≤2;

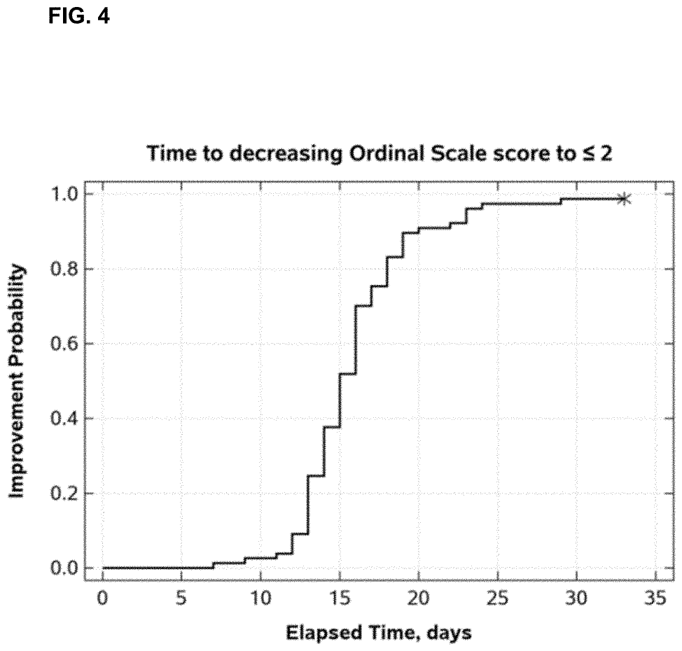

FIG. 5 is another graph that depicts the Evolution of Ordinal Scale score and time-to-event analysis during the treatment and follow-up phases described in Example 1, presented overall, and shows the improvement probability curve plotted for time to decreasing Ordinal Scale score to ≤2 stratified by baseline NEWS severity: ‘Very Severe’ (score≥9); ‘Severe or Moderate’ (score 5-8); and ‘Mild’ (score≤4);

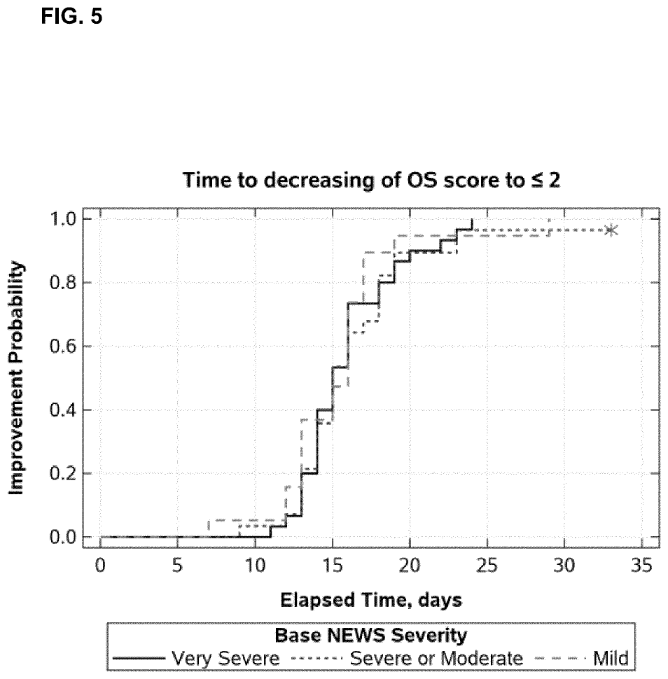

FIG. 6 is another graph that depicts the Evolution of Ordinal Scale score and time-to-event analysis during the treatment and follow-up phases described in Example 1, presented overall, and shows the improvement probability curve plotted for time to decreasing Ordinal Scale score to ≤2 stratified by patient age: <65 years (n=67), ≥65 years (n=10).

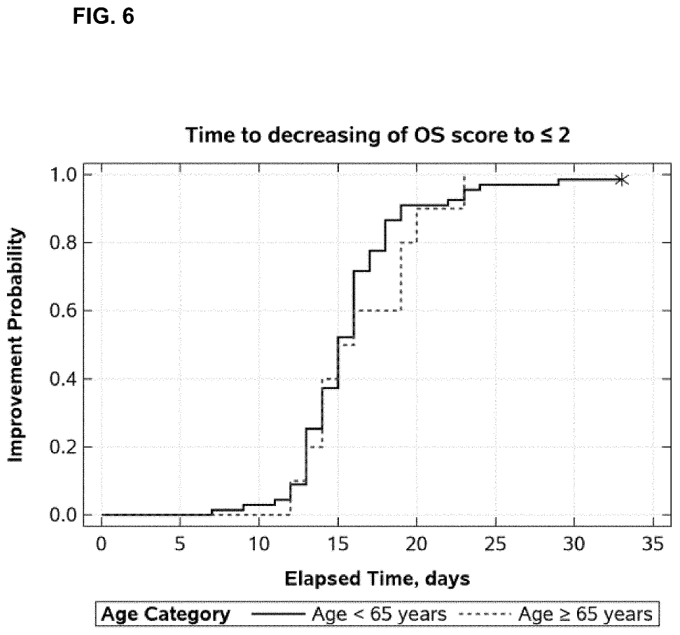

FIG. 7 is a graph that depicts the Evolution of National Early Warning Score (NEWS) values and time-to-event analysis during the treatment and follow-up phases described in Example 1, and shows the mean NEWS during treatment phase;

FIG. 8 is another graph that depicts the Evolution of NEWS values and time-to-event analysis during the treatment and follow-up phases described in Example 1, presented overall, and shows the mean NEWS during treatment phase stratified by baseline NEWS severity: ‘Very Severe’ (score≥9); ‘Severe or Moderate’ (score 5-8), and ‘Mild’ (score≤4);

FIG. 9 is another graph that depicts the Evolution of NEWS values and time-to-event analysis during the treatment and follow-up phases described in Example 1, presented overall, and shows the Mean Ordinal Scale scores during treatment phase stratified by patient age: <65 years and ≥65 years;

FIG. 10 is another graph that depicts the Evolution of NEWS values and time-to-event analysis during the treatment and follow-up phases described in Example 1, presented overall, and shows the improvement probability curve plotted for time to decreasing NEWS to ≤2;

FIG. 11 is another graph that depicts the Evolution of NEWS values and time-to-event analysis during the treatment and follow-up phases described in Example 1, presented overall, and shows the improvement probability curve plotted for time to decreasing NEWS to ≤2 stratified by baseline NEWS severity: ‘Very Severe’ (score≥9); ‘Severe or Moderate’ (score 5-8); ‘Mild’ (score≤4);

FIG. 12 is another graph that depicts the Evolution of NEWS values and time-to-event analysis during the treatment and follow-up phases described in Example 1, presented overall, and shows the improvement probability curve plotted for time to decreasing NEWS to ≤2 stratified by patient age: <65 years, ≥65 years;

FIG. 13 is a graph that depicts the oxygen saturation over time during the treatment phase described in Example 1, presented overall, and shows the mean blood saturation over time during treatment phase;

FIG. 14 is another graph that depicts the oxygen saturation over time during the treatment phase described in Example 1, presented overall, and shows the mean blood saturation over time during treatment phase stratified by baseline NEWS severity: ‘Very Severe’ (score≥9); ‘Severe or Moderate’ (score 5-8); ‘Mild’ (score≤4);

FIG. 15 is another graph that depicts the oxygen saturation over time during the treatment phase described in Example 1, presented overall, and shows the mean blood saturation over time during treatment phase stratified by patient age: <65 years and ≥65 years;

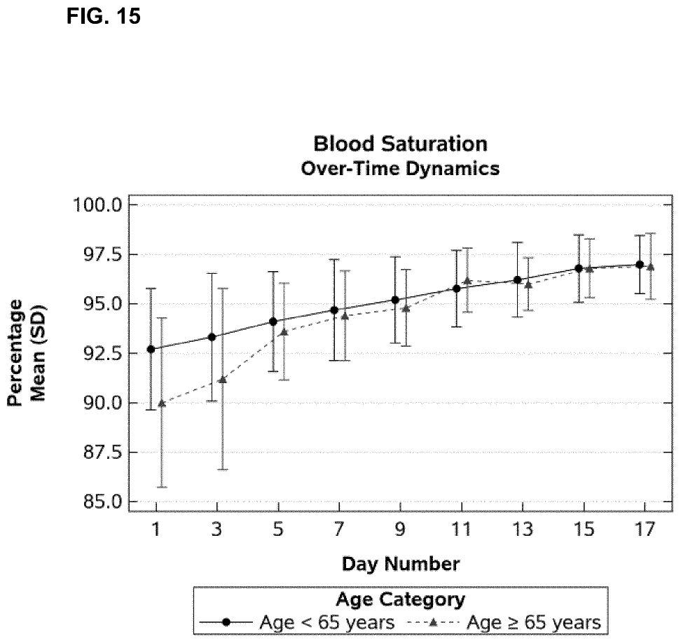

FIG. 16 is a graph that depicts the improvement probability curves plotted for time to vanishing signs of pneumonia of patients treated in Example 1, presented overall, and shows the improvement probability curve plotted for time to vanishing signs of pneumonia;

FIG. 17 is another graph that depicts the improvement probability curves plotted for time to vanishing signs of pneumonia of patients treated in Example 1, presented overall, and shows the improvement probability curve plotted for time to vanishing signs of pneumonia stratified by baseline NEWS severity: ‘Very Severe’ (score≥9); ‘Severe or Moderate’ (score 5-8); ‘Mild’ (score≤4);

FIG. 18 is another graph that depicts the improvement probability curves plotted for time to vanishing signs of pneumonia of patients treated in Example 1, presented overall, and shows the improvement probability curve plotted for time to vanishing signs of pneumonia stratified by baseline age category: <65 years and ≥65 years (note: crosses on the graphs denote censored observations where further data from the patients were not available);

FIG. 19 is a graph that depicts the evolution of body temperature over time and time-to-event analysis during the treatment and follow-up phases described in Example 1, presented overall, and shows the mean body temperature over time during treatment phase;

FIG. 20 is another graph that depicts the evolution of body temperature over time and time-to-event analysis during the treatment and follow-up phases described in Example 1, presented overall, and shows the mean body temperature over time during treatment phase stratified by baseline NEWS severity: ‘Very Severe’ (score≥9), ‘Severe or Moderate’ (score 5-8), and ‘Mild’ (score≤4);

FIG. 21 is another graph that depicts the evolution of body temperature over time and time-to-event analysis during the treatment and follow-up phases described in Example 1, presented overall, and shows the improvement probability curve plotted for time to decreasing body temperature to ≤37° C.; and

FIG. 22 is a graph that depicts the C-reactive protein values over time during the treatment phase described in Example 1.

### DETAILED DESCRIPTION

Severe respiratory failure in patients with COVID-19 is associated with complex immune dysregulation or macrophage activation. Immune dysregulation, mediated by overproduction of interleukin-6 (IL-6), compromises viral clearance. Rapid shedding of endogenous IL-6 receptor (IL-6R) occurs during neutrophil pyroptosis, affecting trans-signaling by augmenting the soluble IL-6R (sIL-6R)/IL-6 complex, and stimulating endothelial cells and ultimately increasing the inflammatory response. Interleukin-6 blocks dendritic cell (DC) maturation, which can prevent induction of T-cell differentiation. The contribution of immune dysfunction to the progression of COVID-19 attracted the inventors of the present application to look for immunological interventions for treating COVID-19.

Azoximer bromide (AZB, Polyoxidonium®) is a macromolecular compound with immunomodulating properties, which was described in U.S. Pat. No. 5,503,830 (incorporated by reference herein) to be effective to treat acute and chronic viral and bacterial infections (e.g., influenza virus), as well as for certain other indications including treatment of immunodeficiencies (e.g., infection prophylaxis in rheumatoid arthritis patients taking immunosuppressant medications). AZB is generally well-tolerated by human subjects with no major safety concerns.

In vitro studies have shown that AZB can induce T-cell proliferation, increase natural killer cell degranulation, and increase immature DC expansion (notably, certain DC costimulatory molecules that stimulate T-cells proliferate following AZB administration). After penetrating leukocytes by endocytosis, AZB is known to localize in cytosolic endoplasmic vesicles, resulting in significant dose-dependent increases in intracellular hydrogen peroxide, which plays a role in the activation of nuclear factor (NF)-κB, which subsequently regulates transcription of genes involved in the inflammatory and immune responses, thereby coordinating several facets of the immune system necessary for infection resistance. There are no current indications for administering AZB to patients afflicted with COVID-19.

Generally, the present inventors unexpectedly discovered that azoximer bromide (AZB) can treat COVID-19 infection when administered to infected individuals by both reducing the severity of COVID-19 symptoms, as well as reducing the overall sickness period. In addition, the present inventors unexpectedly discovered that AZB can effectively prevent COVID-19 infection when administered as a prophylaxis to people who are not afflicted by COVID-19. Notably, as can be seen below, some of the exemplary methods of administration of AZB (i.e., as defined by days and intervals of treatment, dosages, and mode of administration (e.g., pill, injection, spray, suppository, etc.)) are described below as being used for treating COVID-19, some of the exemplary methods of administration of AZB are described below as being used for prophylaxis against COVID-19, and some of the exemplary methods of administration of AZB are shown below as being used both as treatment of patients infected with COVID-19 and as prophylaxis against COVID-19 (when administered to people not yet infected with COVID-19).

With respect to treatment, in some embodiments, a method of treating COVID-19 includes administering, to a person infected with COVID-19, a therapeutically effective amount of azoximer bromide. Notably, azoximer bromide may be advantageously administered as a treatment for COVID-19 in combination with one or more other therapeutic agents. For example, in some embodiments, azoximer bromide is administered as a therapy to patients infected with COVID-19 in combination with one or more of vitamins (e.g., vitamin C), minerals (e.g., zinc), antivirals (e.g., umifenovir, Oseitamivir, tilorone, pentanedioic acid imidazolyl ethanamide, etc.), antibiotics (e.g., doxycycline, etc.), immunostimulants/immunotherapeutics (e.g., interferons, etc.) anticoagulants, hydroxychloroquine, mucolytics, and other drugs, as appropriate. As discussed in more detail below, administration of AZB as part of a complex treatment of COVID-19-afflicted individuals in combination with at least one of a vitamin, mineral, antibiotic, antiviral, immunostimulant, anti-malarial, and mucolytic has been shown to be significantly more effective than the same complex treatment without AZB, highlighting the fact that AZB alone or in combination with other drugs/supplements may be advantageously used to treat COVID-19.

In one embodiment, the method of treatment includes injecting 12 mg of azoximer bromide intravenously once per day on days 1, 2, and 3 of treatment, and then intramuscularly on day 5 and every other day thereafter (i.e., on days 7, 9, 11, 13, 15, and 17) for a total of 10 injections. An alternative method of treating COVID-19 infections in affected individuals includes injecting 6 mg of azoximer bromide intramuscularly once per day on days 1, 2, and 3 of treatment, and then every other day thereafter (i.e., on days 7, 9, 11, 13, 15, and 17) until day 17, for a total of 10 injections.

The method of treatment of COVID-19 is not limited to injections only. For example, in some aspects, a method of treatment of COVID-19 includes administering, to a person infected with COVID-19, 12 mg tablets of azoximer bromide twice per day for 7 consecutive days. In some embodiments the tables are sublingual, but it will be appreciated that AZB tablets meant to be chewed or swallowed may be used instead, when appropriate. In other embodiments, methods of treatment against infection with COVID-19 includes administering, to a subject who is infected with COVID-19, a therapeutically effective amount of azoximer bromide by administering one 12 mg tablet of azoximer bromide twice per day for 10, 20, or 30 consecutive days.

In another variation, a method of treatment of COVID-19 infections in affected individuals includes spraying 6 mg of azoximer bromide intranasally (e.g., three drops into each nostril) three times per day for 10 consecutive days. In yet another aspect, a method of treatment of COVID-19 infections in affected individuals includes administering a 12 mg suppository of azoximer bromide once per day for 10 consecutive days. Notably, intranasal sprays and/or suppositories may be used in patients with severe intoxication and/or vomiting, as well as in cases where it is not practical to use injectable forms.

With respect to prophylaxis, in some embodiments, a method of prophylaxis against infection with COVID-19 includes administering, to a healthy subject who is not infected with COVID-19, a therapeutically effective amount of azoximer bromide. In one embodiment, the prophylaxis is administered by injecting 12 mg of azoximer bromide intravenously once per day on days 1, 2, and 3 of prophylaxis, and then intramuscularly on day 5 and then every other day thereafter (i.e., on days 7, 9, 11, 13, 15, and 17) until day 17, for a total of 10 injections. In another embodiment, a method of prophylaxis against infection with COVID-19 includes administering, to a healthy subject who is not infected with COVID-19, a therapeutically effective amount of azoximer bromide by injecting 6 mg of azoximer bromide intramuscularly once per day on days 1, 2, and 3 of prophylaxis, and then on day 5 and then every other day thereafter (i.e., on days 7, 9, 11, 13, 15, and 17) until day 17, for a total of 10 injections.

As with the method of treatment, the prophylaxis for COVID-19 does not have to be necessarily administered by way of injections. For example, in one embodiment, a method of prophylaxis against infection with COVID-19 includes administering, to a healthy subject who is not infected with COVID-19, a therapeutically effective amount of azoximer bromide by administering one 12 mg tablet of azoximer bromide once per day for 10 consecutive days.

In other embodiments, methods of prophylaxis against infection with COVID-19 includes administering, to a healthy subject who is not infected with COVID-19, a therapeutically effective amount of azoximer bromide by administering one 12 mg tablet of azoximer bromide once per day for 3, 5, 7, 8, 20, or 30 consecutive days. In another embodiment, a method of prophylaxis against infection with COVID-19 includes administering, to a healthy subject who is not infected with COVID-19, a therapeutically effective amount of azoximer bromide by administering one 12 mg tablet of azoximer bromide twice per day for 10 consecutive days. In yet another embodiment, a method of prophylaxis against infection with COVID-19 includes administering, to a healthy subject who is not infected with COVID-19, a therapeutically effective amount of azoximer bromide by administering two 12 mg tablets of azoximer bromide once per day for 10 consecutive days In still another embodiment, a method of prophylaxis against infection with COVID-19 includes administering, to a healthy subject who is not infected with COVID-19, a therapeutically effective amount of azoximer bromide by administering one 12 mg tablet of azoximer bromide three times per day for 10 consecutive days.

In another exemplary embodiment, a method of prophylaxis against infection with COVID-19 includes administering, to a healthy subject who is not infected with COVID-19, a therapeutically effective amount of azoximer bromide by spraying 6 mg of azoximer bromide intranasally (e.g., three drops into each nostril) three times per day for 10 consecutive days. In yet another exemplary embodiment, a method of prophylaxis against infection with COVID-19 includes administering, to a healthy subject who is not infected with COVID-19, a therapeutically effective amount of azoximer bromide by administering a 12 mg suppository of azoximer bromide once per day for 10 consecutive days.

Advantages and embodiments of the methods and products described herein are further illustrated by the following examples; however, the particular conditions, processing schemes, materials, and amounts thereof recited in these examples, as well as other conditions and details, should not be construed to unduly limit these methods and products. The following examples illustrate the advantages of AZB in treating individuals infected with COVID-19 (Example 1) and as a prophylaxis against COVID-19 (Example 2) according to the methods described herein.

### EXAMPLE 1

The AZB-based COVID-19 treatment method described herein was tested in an open-label, multi-center study in patients hospitalized due to COVID-19 that was conducted between March 2020 and July 2020 at several hospitals in the Russian Federation and one hospital in Belarus. The study was exploratory in nature and therefore did not include a placebo group. The study was conducted in compliance with the International Council for Harmonisation harmonized tripartite guidelines regarding Good Clinical Practice and the principles enshrined in the Declaration of Helsinki, the Medicines for Human Use (Clinical Trials) Regulations, as well as the standards set out by the Research Governance Framework, and all local laws and regulations. The study was approved by two Independent Ethical Committees (Pharmnadzor and Local Ethical Committee of the Grodno Regional Infectious Disease Clinical Hospital).

Generally, after a screening period (Day −1 to Day 1), eligible subjects that were hospitalized as a result of COVID-19 infection were administered AZB for 17 days (Day 1 to Day 17). Patients were monitored during a planned follow-up period between Day 18 and Day 29±3. In some patients, the final follow-up visit was performed up to Day 73.

Adults aged 18 years and over and hospitalized due to COVID-19 symptoms with a laboratory confirmed SARS-CoV-2 infection were enrolled in the study after providing informed consent. Infection of the patients with COVID-19 was determined by polymerase chain reaction (PCR) or other commercially available assays from a specimen collected from the patients before enrolment in the study. Patients who were pregnant/breastfeeding, displayed increased sensitivity to any component of the study treatment, had acute/chronic renal failure, or exhibited pathological conditions judged to make study participation unwarranted were excluded from the study.

Demographic data and detailed medical history were collected from the patients after obtaining informed consent. Eligible patients received 12 mg AZB (lyophilizate for solution for injections and topical application, manufactured by NPO Petrovax Pharm, Moscow, Russia) intravenously once daily on Day 1, Day 2, and Day 3, and then intramuscularly every other day from Day 5 to Day 17 (inclusive) for a total of 10 injections over the 17 day period. In addition to this 17 day AZB treatment, the test patients also received standard of care (SOC) treatment for COVID-19 in accordance with existing clinical recommendations of the Ministry of Health of the Russian Federation including but not limited to one or more of: certain antibiotics, antivirals, anticoagulants, hydroxychloroquine, other drugs, and vitamins as appropriate.

The requirement of patients for oxygen therapy, high flow oxygen devices, noninvasive ventilation, mechanical ventilation (via an endotracheal tube or tracheostomy tube), or extracorporeal membrane oxygenation was assessed daily. Other daily assessments included clinical status as determined by a seven-point World Health Organization (WHO)-recommended Ordinal Scale (OS), shown in Table 1 below.

TABLE 1
Seven-point Ordinal Scale According to the WHO Master Protocol

| Ordinal score | Event |
|---|---|
| 1 | Not hospitalized, no limitations on activities |
| 2 | Not hospitalized, limitation on activities |
| 3 | Hospitalized, not requiring supplemental oxygen |
| 4 | Hospitalized, requiring supplemental oxygen |
| 5 | Hospitalized, on noninvasive ventilation or high flow |
| | oxygen devices |
| 6 | Hospitalized, on invasive mechanical ventilation or |
| | extracorporeal membrane oxygenation |
| 7 | Death |

Disease severity of the patients was determined according to the seven-parameter National Early Warning Score (NEWS), which includes (1) respiration rate, (2) oxygen saturation, (3) supplemental oxygen, (4) temperature, (5) systolic blood pressure, (6) heart rate, and (7) level of consciousness. NEWS parameters were also measured individually as absolute values. Other assessments included: safety laboratory tests (including C-reactive protein [CRP]); physical examination; clinical signs and symptoms; electrocardiogram; evaluation of chest X-ray/computed tomography (CT) scans; nasopharyngeal and/or oropharyngeal smear assessment for polymerase chain reaction; and bacteriological sputum culture. Adverse events (AEs) and serious adverse events (SAEs) were graded according to The Division of AIDS Table for Grading the Severity of Adult and Pediatric Adverse Events, version 2.1. The patients were monitored after hospital discharge (at least until Day 29) until completion of an End-of-Study assessment. Protocol deviations were recorded and classified as either significant or non-significant.

The protocol-defined primary endpoint was patient clinical status (according to OS) at Day 15 compared with baseline. Exploratory analyses included clinical severity as determined by the seven-point OS and NEWS values, hospitalization duration, signs of pneumonia, body temperature, and oxygen saturation (SpO₂).

Pneumonia presence was determined according to the following criteria. Pneumonia was considered present during a visit if: (1) positive signs were observed on an X-ray/CT scan; (2) no positive signs on the X-ray/CT scan were noted, but the visit lay between positive scans obtained on other days; and (3) the visit was after the last recorded positive X-ray/CT scan OR no X-ray/CT scans were done for the patient during the study (one of the following must also have applied: respiratory support was used, blood saturation was below 96%, respiratory rate was above 20, dyspnea was present, cough was present). In all other cases, pneumonia was considered absent.

The patients were assigned COVID-19 severity groups based on their baseline NEWS: ‘Very Severe’ (NEWS≥9), ‘Severe or Moderate’ (NEWS 5-8), or ‘Mild’ (NEWS≤4). The time-to-event analysis threshold was set to NEWS≤2 (equating to hospital discharge). For the over-time dynamics analysis, discharged patients were treated as having an OS score of 2 commencing the day of discharge until an OS score was measured at follow-up. Outcome measures for safety analysis included the cumulative incidence of AEs/SAEs and adverse reactions (ARs)/serious adverse reactions (SARs) and the assessment of laboratory parameters. Permanent or temporary discontinuation of infusions and/or injections was documented.

Statistical analyses were performed using SAS® version 9.4 (SAS Institute Inc., Cary, N.C., USA). Safety analyses were performed on the Intent-to-Treat (ITT) population, comprising all patients who received at least one dose of investigational product and had at least one valid post-baseline value for primary endpoint evaluation. All other analyses were performed on the Per Protocol (PP) population.

Safety outcome measures were presented using frequency counts and percentages. Quantitative data were summarized using descriptive statistics (arithmetic mean and standard deviation). Qualitative data were presented using incidences, percentages, or proportions. The significance level was 0.05 with 95% confidence intervals presented in all analyses. Survival analysis for time-to-event data was performed via the use of Kaplan-Meier curves. The Student t-test (for dependent or independent samples) and analysis of variance (for repeated measurements) were the standard parametric tests used for the comparison of quantitative data with normal distribution. Non-parametric Mann-Whitney U, Wilcoxon, and Friedman tests were used for the comparison of non-normally distributed quantitative data. Normality of distribution was assessed by the Shapiro-Wilk test and incidences were compared with the Pearson's chi-squared test or Fischer's exact test.

To address the absence of a direct control group, trends in a control group of patients with COVID-19 receiving SOC treatment from a previously published randomized, controlled, open-label trial study were analyzed. Such a was identified by way of a structured literature search via PubMed (the search aimed to find clinical trials conducted in patients with COVID-19 with a control arm comprising SOC treatment only), and was selected due to the alignment of several outcome measures used in the present study. This study was conducted at a single center in China between January 2020 and February 2020, and included 199 patients hospitalized with a confirmed SARS-CoV-2 diagnosis. Patients received lopinavir-ritonavir (400 mg and 100 mg, respectively) twice daily plus SOC for 14 days or SOC alone. The primary endpoint was time to clinical improvement as determined by the same seven-point OS used in the present study. Differences in basic baseline demographics (age, sex, baseline OS score), OS score at the end of study duration, duration of hospitalization, and Day 28 mortality between the two patient groups (AZB or control) were assessed.

In the present study, eighty-one patients were eligible for study inclusion and comprised the ITT population. Eighty patients participated in the study. Four patients with very mild COVID-19 symptoms were incorrectly hospitalized and were excluded from the ITT population, since these patients did not require hospital treatment (antibiotics, oxygen support, etc.), had low baseline OS and NEWS values, and recovered by themselves in the absence of medical intervention. Therefore, seventy-seven (77) patients therefore comprised the per protocol (PP) population. With the exception of safety analyses, all results are presented for the Population ([n=77]). The median age of the patients was 53.0 years (range: 22-81 years) and 54.5% of patients were male (Table 2).

TABLE 2
Demographic and baseline characteristics (Intent-to-Treat (ITT) and Per
Protocol (PP) populations)

| | ITT Population | PP Population |
|---|---|---|
| | N = 81 | N = 77 |
| Age, years | | |
| Mean | 50.88 | 52.32 |
| SD | 12.81 | 11.37 |
| Median | 53.0 | 53.0 |
| Min, Max | 18.0, 81.0 | 22.0, 81.0 |
| Age categories, n (%) | | |
| <65 years | 71 (87.7) | 67 (87.0) |
| ≥65 years | 10 (12.3) | 10 (13.0) |
| <60 years | 57 (70.4) | 53 (68.8) |
| ≥60 years | 24 (29.6) | 24 (31.2) |
| Sex, n (%) | | |
| Male | 44 (54.3) | 42 (54.5) |
| Female | 37 (45.7) | 35 (45.5) |
| Ordinal Scale score, n | | |
| Mean | 4.30 | 4.36 |
| SD | 1.02 | 1.00 |
| NEWS, n | | |
| Mean | 6.51^{a} | 6.86^{b} |
| SD | 3.41^{a} | 3.14^{b} |
| Severity according to NEWS, n (%) | | |
| Very Severe (NEWS ≥ 9) | 30 (37.0) | 30 (39.0) |
| Severe or Moderate (NEWS 5-8) | 28 (34.6) | 28 (36.4) |
| Mild (NEWS ≤ 4) | 23 (28.4) | 19 (24.7) |
| Signs of pneumonia, n (%) | | |
| Yes | 77 (95.1) | 74 (96.1) |
| No | 4 (4.9) | 3 (3.9) |
| Oxygen saturation (SpO₂), % | | |
| Mean | 92.71^{c} | 92.39^{d} |
| SD | 3.53^{c} | 3.32^{d} |
| Respiratory support requirement, n (%) | | |
| Present | 59 (72.8) | 59 (76.6) |
| Absent | 22 (27.2) | 18 (23.4) |
| Respiratory support type, n (%) | | |
| Invasive lung ventilation | 11 (13.6) | 11 (14.3) |
| Noninvasive lung ventilation or high-flow | 24 (29.6) | 24 (31.2) |
| oxygen devices | | |
| Oxygen therapy | 24 (29.6) | 24 (31.2) |
| None | 22 (27.2) | 18 (23.4) |
| Time from disease onset to treatment initiation, | | |
| days | | |
| Mean | 6.79^{e} | 7.05^{f} |
| SD | 5.16^{e} | 5.17^{f} |
| Duration of hospitalization, days | | |
| Mean | 19.13^{g} | 19.32^{h} |
| SD | 5.29^{g} | 5.33^{h} |
| Min, Max | 10.0, 38.0^{g} | 10.0, 38.0^{h} |
| Concomitant medication by type, n (%) | | |
| Antivirals | 46 (56.8) | 42 (54.5) |
| Hydroxychloroquine | 37 (45.7) | 37 (48.1) |
| Anticoagulants | 41 (50.6) | 41 (53.2) |
| Body temperature, ° C. | | |
| Mean | 38.09 | 38.14 |
| SD | 1.07 | 1.07 |
| Cough severity, n (%) | | |
| No cough | 8 (9.9) | 7 (9.1) |
| Weak | 48 (59.3) | 45 (58.4) |
| Strong | 25 (30.9) | 25 (32.5) |
| C-reactive protein, mg/L | | |
| Mean | 43.21ⁱ | 45.25^{j} |
| SD | 53.03ⁱ | 53.67^{j} |

^{a}Number of cases/number of missing cases: 80/1
^{b}Number of cases/number of missing cases: 76/1
^{c} Number of cases/number of missing cases: 79/2
^{d}Number of cases/number of missing cases: 75/2
^{e}Number of cases/number of missing cases: 78/3
^{f} Number of cases/number of missing cases: 74/3
^{g}Number of cases/number of missing cases: 80/1
^{h}Number of cases/number of missing cases: 76/1
ⁱNumber of cases/number of missing cases: 79/2
^{j}Number of cases/number of missing case: 75/2

In the 77 patients that were treated, COVID-19 severity was assessed as ‘Very Severe’ in 30 patients (39.0%), ‘Severe or Moderate’ in 28 patients (36.4%) and ‘Mild’ in 19 patients (24.7%). Mean (standard deviation [SD]) baseline OS and NEWS values were 4.36 (1.00) and 6.86 (3.14), respectively. Seventy-four patients (96.1%) displayed signs of pneumonia at baseline and 59 patients (76.6%) required respiratory support. Twenty-four patients (31.2%) required oxygen via a mask, 24 patients (31.2%) required noninvasive ventilation of high-flow oxygen devices and 11 patients (14.3%) required mechanical ventilation. Mean (SD) duration of hospitalization was 19.32 days (5.33; range: 10-38 days). Comorbidities were recorded in 58 patients (75.3%), the most frequently reported of which were patients with diabetes and/or metabolic syndrome (n=25) and arterial hypertension (n=23).

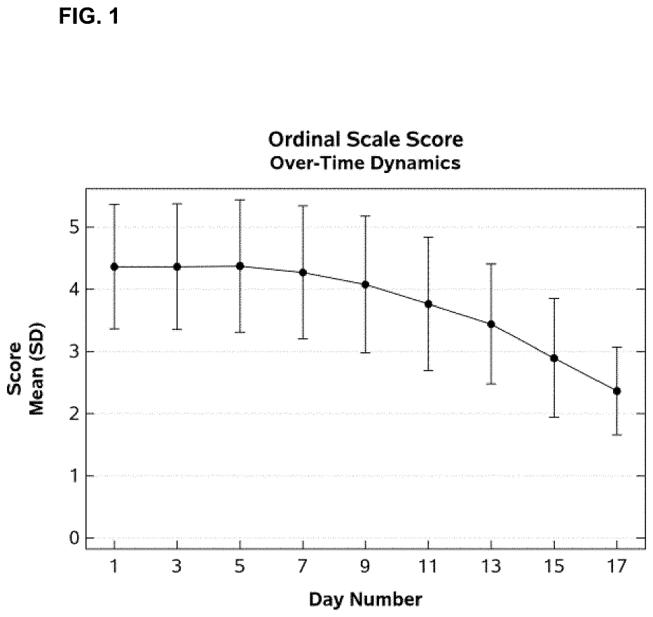

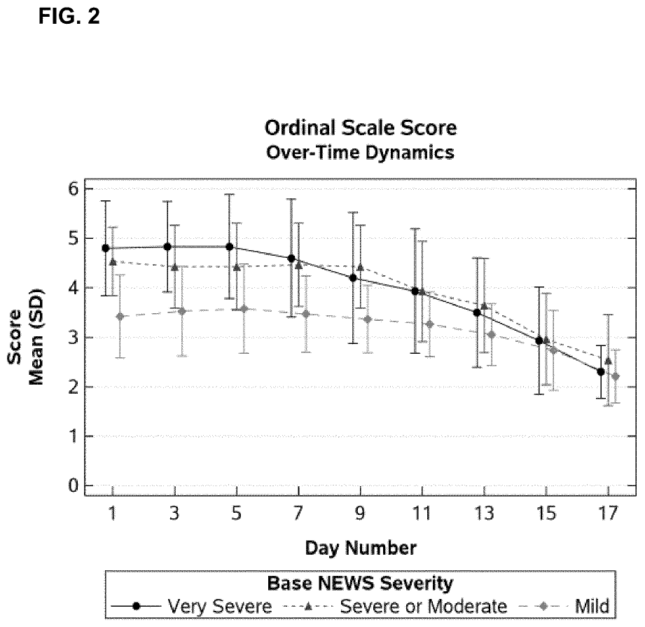

As FIG. 1 shows, the mean (SD) OS scores were relatively stable and close to baseline (4.36 [1.00]) during the first seven days of AZB treatment. The number of patients requiring respiratory support (OS=4-6) was markedly lower on Day 15 (n=14, 18.2%) compared with baseline (n=59, 76.6%). Notable improvements in mean (SD) OS score were observed from Day 9 (4.08 [1.10]) until the end of the treatment period on Day 17 (2.36 [0.71]). This trend continued until the End-of-Study visit (1.12 [0.71]). This improvement was most prominent in patients with a ‘Very Severe’ or ‘Severe to Moderate’ baseline COVID-19 diagnosis, as can be seen in FIG. 2.

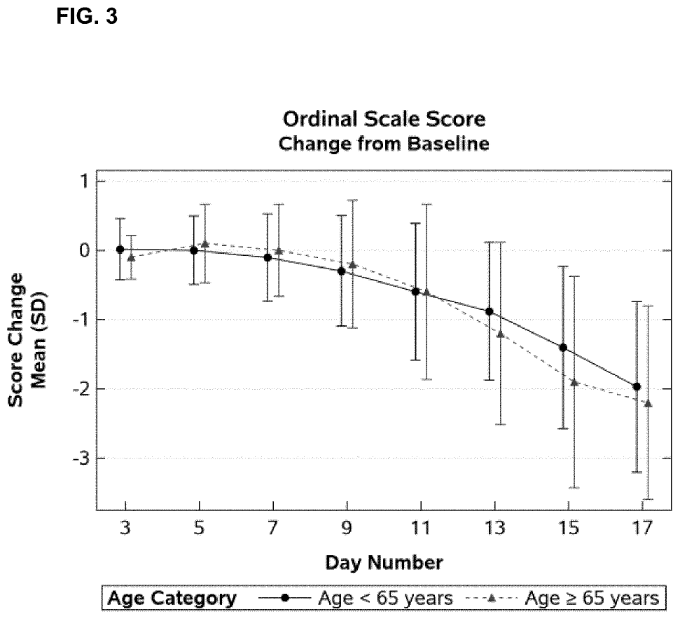

By Day 17, the majority of patients (93.5%; n=72) had an OS score of 2 or 3. Mean (SD) time to an OS score improving to ≤2 was 15.9 days (3.72). Improvements in observed OS scores were similar between patients<65 years and patients≥65 years of age, which can be seen in FIG. 3. Notably, the crosses on the graphs in FIGS. 1-3 denote censored observations, where further data from the patients was not available.

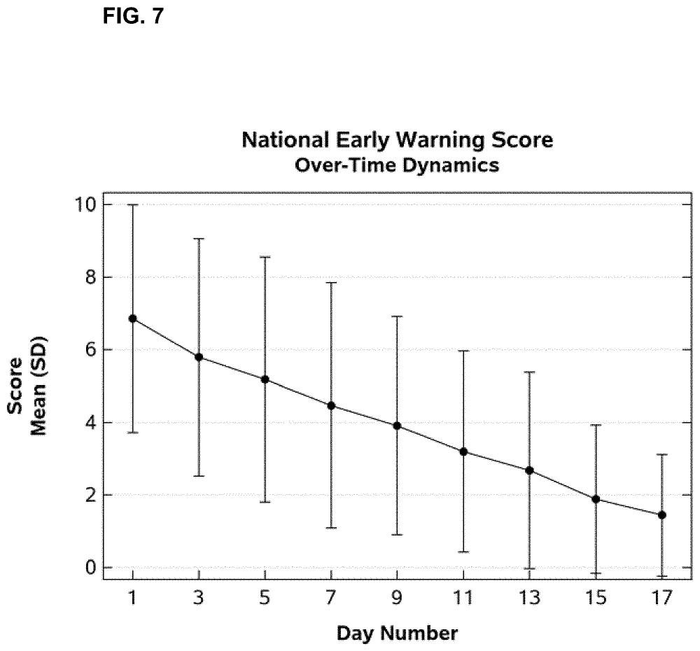

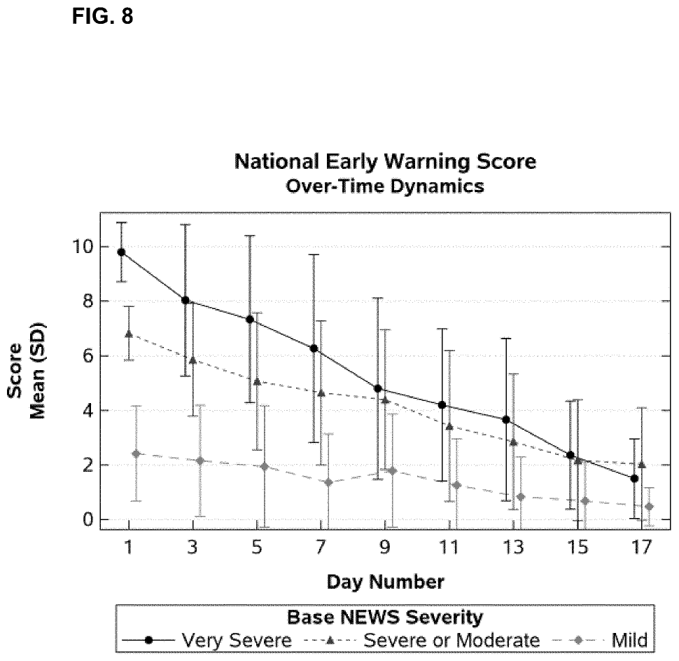

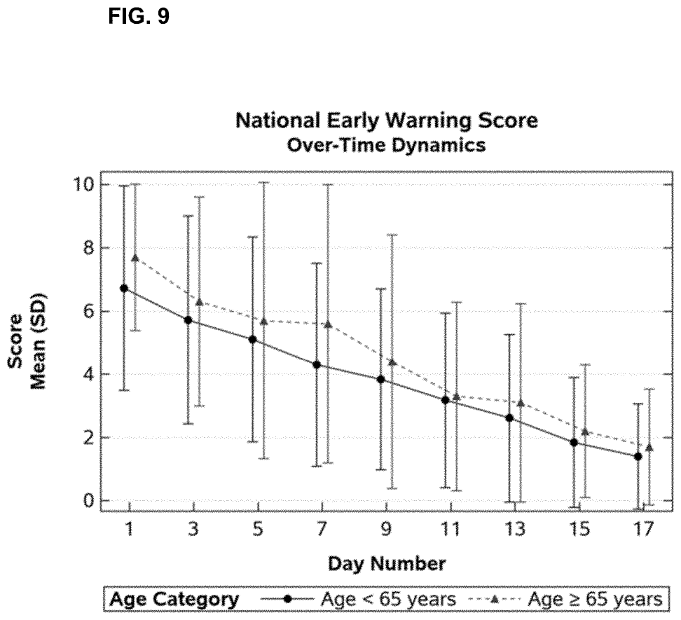

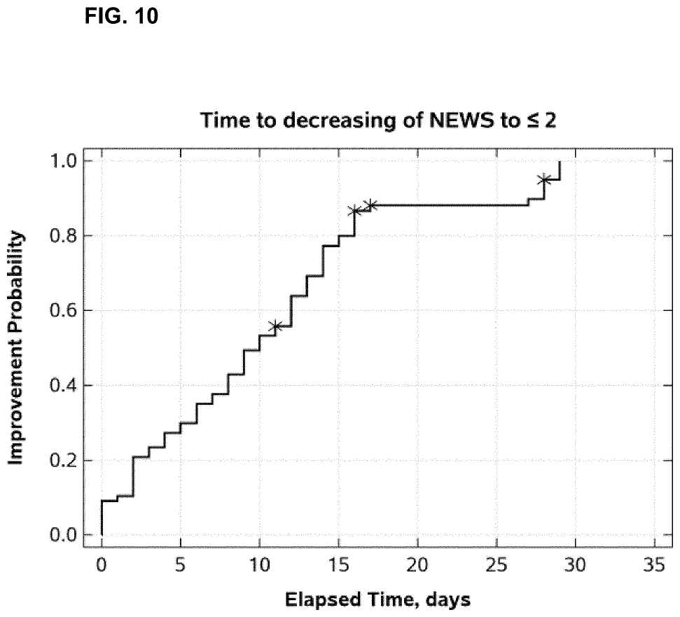

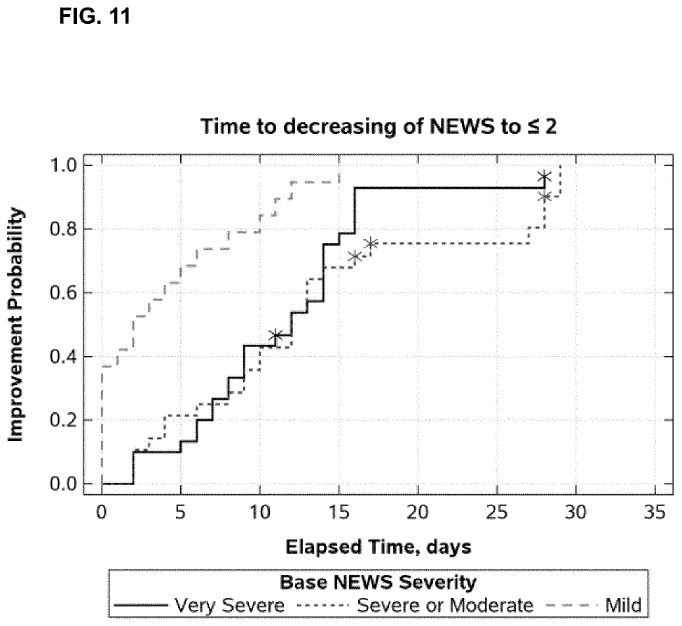

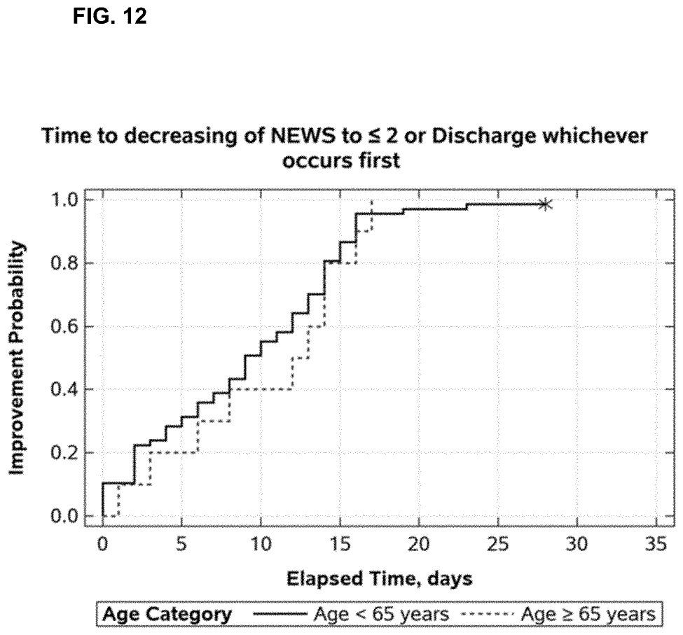

FIG. 7 shows that improvements in mean NEWS were also observed over time and were more notable than the improvements in mean OS scores. The most prominent improvement was also observed in the patients with a ‘Very Severe’ or ‘Severe to Moderate’ baseline COVID-19 diagnosis, as shown in FIG. 8. Patients with a ‘Mild’ baseline COVID-19 diagnosis reached NEWS≤2 quicker than patients in the other two groups (see FIG. 11). However, as shown in FIG. 8, all patient groups reached similar scores by Day 17. Mean (SD) NEWS improved from 6.86 (314) at baseline to 1.08 (1.18) by the End-of-Study visit with an improvement probability of >80% after 15 days of treatment in all patients. Similar improvements in NEWS were observed between patients<65 years and patients≥65 years of age (see FIG. 9) and time to discharge was similar between the two groups (see FIG. 12). Again, the crosses on the graphs in FIGS. 7-12 denote censored observations where further data from the patients were not available.

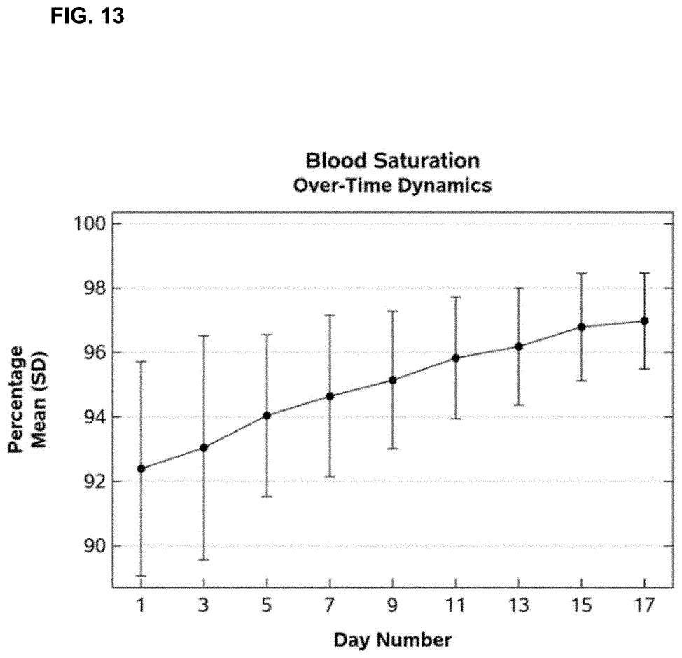

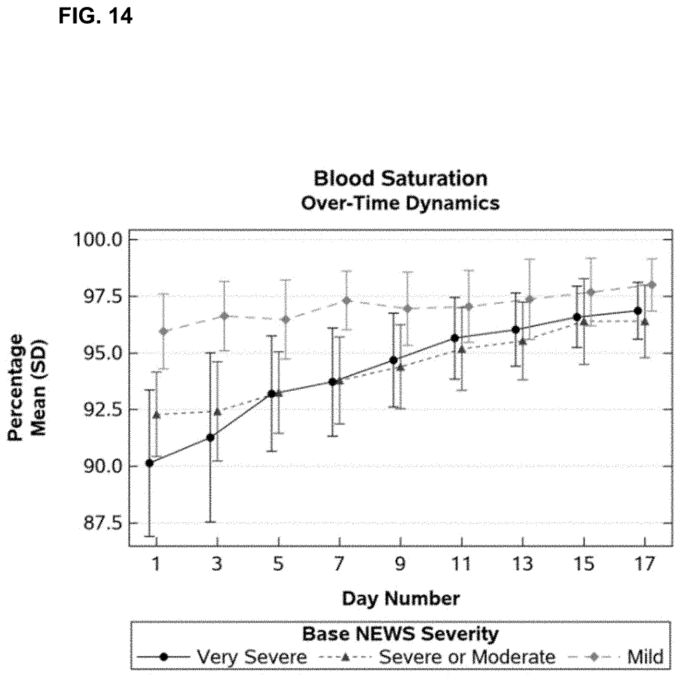

As can be seen in FIG. 13, blood oxygen saturation (SpO₂) steadily improved over the treatment period. The improvement was most prominent in patients with a ‘Very Severe’ or ‘Severe to Moderate’ baseline COVID-19 diagnosis, as shown in FIG. 14, with similar oxygen saturation values observed between these two groups from Day 3 to Day 17. One patient had a slightly low SpO₂ value of 94% at the study completion visit (Day 29±3). The values for all other patients were normal (reference range: 95-99%). Respiratory support requirement (OS=4-6) improved substantially from 59 patients (76.6%) at baseline to 14 patients (18.2%) at Day 15. No patients required respiratory support by their End-of-Study assessment. Mean (SD) oxygen saturation was lower in patients≥65 years of age at baseline (90.00% [4.27]) than patients<65 years of age (92.71% [3.07] (see FIG. 14). However, oxygen saturation reached similar levels in both groups by Day 17 (>65 years: 96.90% [1.66]; <65 years: 96.98% [1.47]).

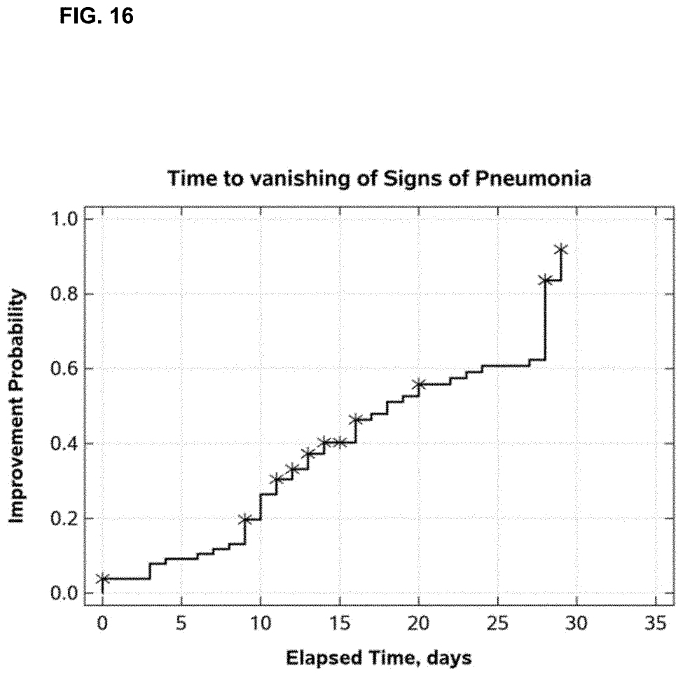

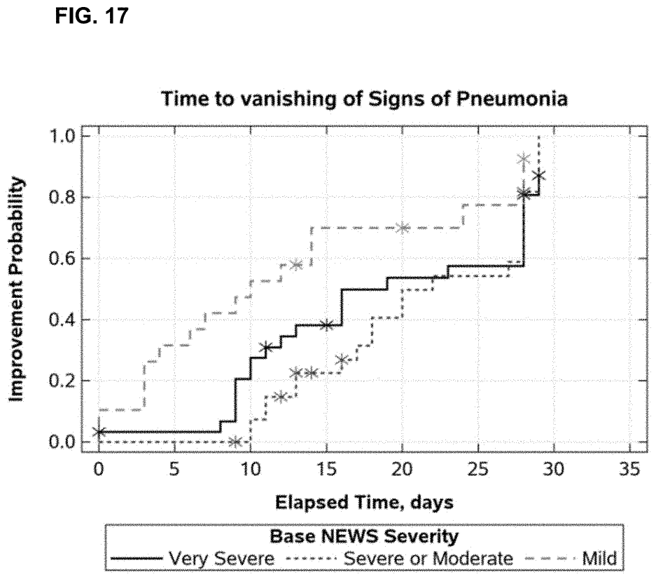

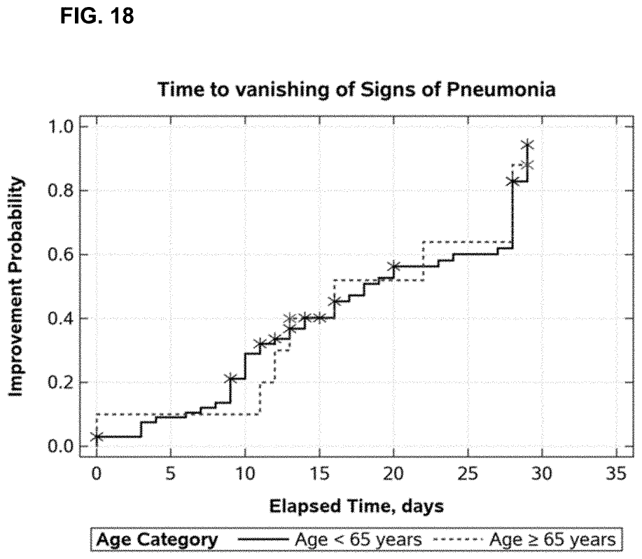

Pneumonia presence in 74 patients (96.1%) at baseline gradually reduced to 18 patients (23.4%) by the End-of-Study assessment. Mean (SD) time to vanishing signs of pneumonia was 18.45 days (9.65). Standard improvement probability curves are shown in FIGS. 16-18. The highest recovery probability was in patients with ‘Mild’ COVID-19 at baseline (see FIG. 17), suggesting a more rapid recovery. The lowest recovery probability was in the ‘Severe or Moderate’ group. Patients<65 years and ≥65 years of age had a similar recovery probability (see FIG. 18). Patients<65 years of age requiring respiratory support (OS=4-6) decreased from 49 patients (73.1%) at baseline to 3 patients (4.5%) by the end of the treatment period. In patients≥65 years of age, this decreased from 10 patients (100.0%) to 1 patient (10.0%).

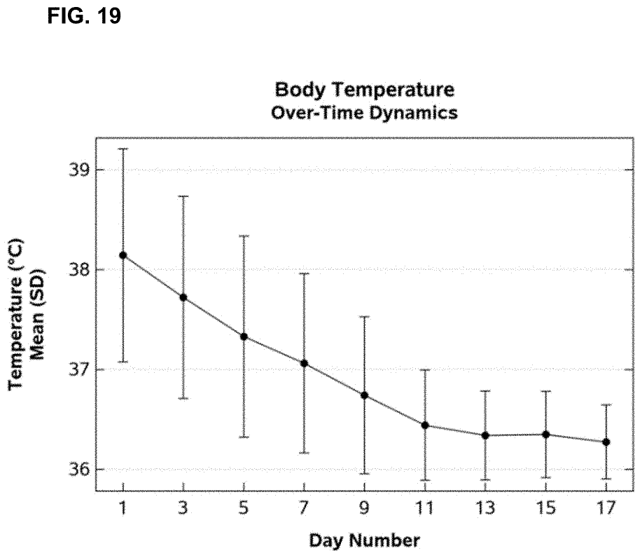

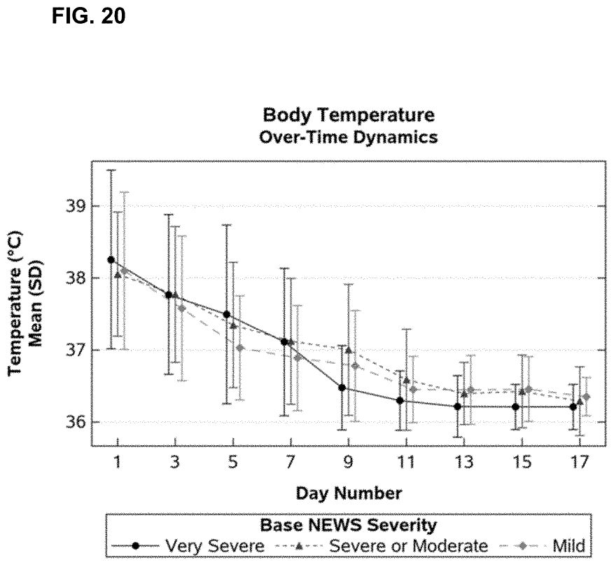

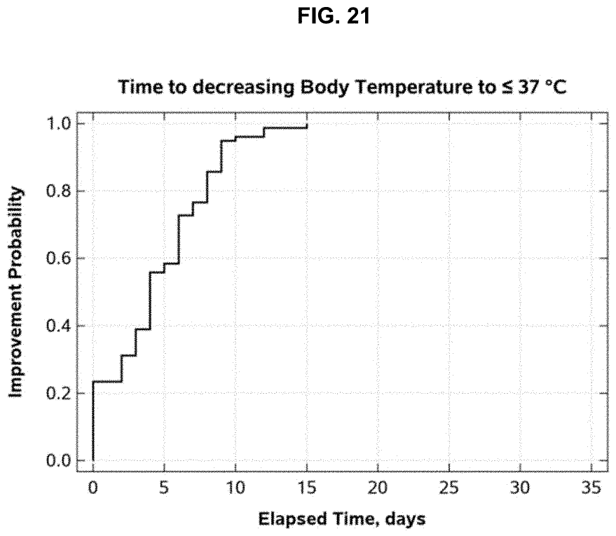

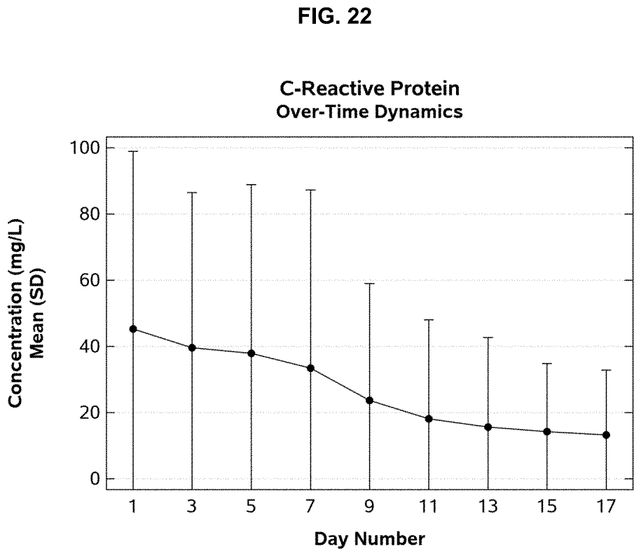

With reference to FIG. 19, most patients had elevated body temperature (>37° C.) at baseline. Notably, as shown in FIG. 20, all patients had a normal body temperature (≤37° C.) by Day 11 and no notable differences were observed when stratified by baseline NEWS severity score. In addition, the probability of achieving a normal body temperature after 10 days of treatment was 90%, as can be seen in FIG. 21. The mean (SD) and median C-reactive protein (CRP) decreased steadily from 45.25 mg/L (53.67) and 29.0 mg/L at baseline to 13.21 mg/L (19.56) and 8.5 mg/L by Day 17, respectively (see FIG. 22).

The historical control group (n=100) from another study the study conducted by Cao et al. (Cao B, Wang Y, Wen D, et al., A Trial of Lopinavir-Ritonavir in Adults Hospitalized with Severe Covid-19. N Engl J Med. 2020; 382(19):1787-1799) had comparable baseline demographics to the patients receiving AZB (see Table 3 below). Table 3 reproduced below compares the demographic, baseline, and efficacy endpoints of the study described above to the historical control group patient data described in Cao et al.

TABLE 3
Comparison of demographic, baseline, and efficacy endpoints with
historical control group patient data (Per Protocol population)

| | | Standard |
|---|---|---|
| | AZB | of Care |
| | (Active | (Historical |
| | Treatment) | Control^{a}) |
| | N = 77 | N = 100 |
| Age, years | | |
| Median | 53.0 | 58.0 |
| Min, Max | 22.0, 81.0 | 48.0, 68.0 |
| Sex, n (%) | | |
| Male | 42 (54.5) | 59 (59.0) |
| Female | 35 (45.5) | 41 (41.0) |
| Baseline Ordinal Scale score, n | | |
| Mean | 4.36 | 3.99 |
| Baseline Ordinal Scale score, n (%) | | |
| 1. Not hospitalized, no limitations on | 0 (0.0) | 0 (0.0) |
| activities | | |
| 2. Not hospitalized, limitation on activities | 0 (0.0) | 0 (0.0) |
| 3. Hospitalized, not requiring supplemental | 18 (23.4) | 17 (17.0) |
| oxygen | | |
| 4. Hospitalized, requiring supplemental | 24 (31.2) | 67 (67.0) |
| oxygen | | |
| 5. Hospitalized, on noninvasive ventilation or | 24 (31.2) | 16 (16.0) |
| high flow oxygen devices | | |
| 6. Hospitalized, on invasive mechanical | 11 (14.3) | 0 (0.0) |
| ventilation or extracorporeal membrane | | |
| oxygenation | | |
| 7. Death | 0 (0.0) | 0 (0.0) |
| Day 14 or Day 15^{b} Ordinal Scale score, n | | |
| Mean | 2.90 | 3.87 |
| Day 14 or Day 15^{b} Ordinal Scale | | |
| score, n (%) | | |
| 1. Not hospitalized, no limitations on | 1 (1.3) | |
| activities | | |
| 2. Not hospitalized, limitation on activities | 27 (35.1) | 28 (28.0) |
| 3. Hospitalized, not requiring supplemental | 35 (45.5) | 24 (24.0) |
| oxygen | | |
| 4. Hospitalized, requiring supplemental | 9 (11.7) | 20 (20.0) |
| oxygen | | |
| 5. Hospitalized, on noninvasive ventilation or | 3 (3.9) | 6 (6.0) |
| high flow oxygen devices | | |
| 6. Hospitalized, on invasive mechanical | 2 (2.6) | 5 (5.0) |
| ventilation or extracorporeal membrane | | |
| oxygenation | | |
| 7. Death | 0 (0.0) | 17 (17.0) |
| Day 28 Mortality, n (%) | | |
| Alive | 77 (100.0) | 75 (75.0) |
| Dead | 0 (0.0) | 25 (25.0) |
| Duration of Hospitalization, days | | |
| Median | 19.0^{c} | 16.0 |
| Min, Max | 10.0, 38.0^{c} | 13.0, 18.0 |

^{a}Historical control data was obtained from Cao et al., 2020).
^{b}Recorded on Day 14 for patients receiving standard of care (historical control) and Day 15 for patients receiving AZB (active treatment).
^{c}Number of cases/number of missing cases: 80/1

In the historical control study, the median age was 58.0 years (range: 48.0-68.0 years) with a similarly slightly higher proportion of male patients (59.0%; n=59) to female patients (41.0%; n=41). The mean baseline OS score was slightly higher in the control group (3.99) compared with the AZB group (4.36). The baseline OS scores ranged between 3 and 5 and the majority of patients (67.0%; n=67) had a score of 4. In patients who received AZB, the majority of patients had a score of 3 (23.4%; n=18), 4 (31.2%; n=24), or 5 (31.2%; n=24), while 14.3% (n=11) of patients had a score of 6.

A higher proportion of patients receiving AZB therefore required invasive or noninvasive ventilation (OS 5 or 6) compared with the historical control group. The change in mean OS score from baseline to Day 14/15 was not notable in the historical control group (3.99 to 3.87), but was more pronounced in the AZB group (4.36 to 2.90). By Day 14 in the historical control group, most patients had an OS score of 2 (28.0%; n=28), 3 (24.0%; n=24), or 4 (20.0%; n=20). Six (6.0%) and 5 (5.0%) patients had an OS score of 5 and 6, respectively, while 17 patients (17.0%) had died.

By Day 15 in the AZB group, the majority of patients had an OS score of 2 (35.1%; n=27) or 3 (45.5%; n=35). Three patients (3.9%) and two patients (2.6%) had an OS score of 5 and 6, respectively, one patient (1.3%) had an OS score of 1, and no patients had died. By Day 28, 25 patients (25.0%) in the control group had died compared with no patients in the AZB group. Median duration of hospitalization in the control and AZB groups were similar at 16.0 days (range: 13.0-18.0) and 19.0 days (range: 10.0-38.0), respectively.

With regard to concomitant standard of care (SOC) medications (e.g., antiviral, anticoagulant, and hydroxychloroquine) being used to treat the patients to whom AZB was administered in the present study, the changes in NEWS and OS scores, oxygen saturation (SpO₂), and body temperature were stratified by antiviral, anticoagulant, and hydroxychloroquine usage, as can be seen in Table 2 above. No adverse interactions of the concomitant medications with AZB were observed. Baseline mean (SD) NEWS appeared notably higher in patients who were receiving antivirals (8.17 [2.21]; 42 patients) in comparison to those patients that were not receiving antivirals (5.31 [3.38]; 35 patients). Similarly, the baseline mean (SD) NEWS was higher in patients receiving hydroxychloroquine (5.75 [3.30]; 37 patients) as compared to the patients not receiving hydroxychloroquine (7.85 [2.65]; 40 patients).

Time-to-event analysis showed that mean (SD) time to NEWS decreasing to ≤2 was faster in patients receiving hydroxychloroquine (7.30 days [5.78]) as compared to the patients not receiving hydroxychloroquine (13.47 days [8.81]). The opposite trend was noted in patients receiving antivirals (13.40 days [8.51]) than those not receiving antivirals (7.03 days [5.99]).

In the ITT population (N=81), six patients (7.4%) each experienced one adverse event (AE) of PQ interval prolongation (two events), fever (two events), intermittent fever, and bacterial pneumonia. No adverse events were related to AZB administration and all resolved. One patient who experienced the adverse event of bacterial pneumonia was discontinued from the study after two doses of AZB and required further medication; all other patients completed the study. No deaths were recorded during the study period.

One patient experienced a serious adverse event (SAE) of Klebsiella sepsis and died after study completion (Day 34) due to associated complications including respiratory distress, disease progression, secondary bacterial infection, sepsis, and multiple organ failure. However, the SAE was determined to be unrelated related to treatment with AZB. The patient was 30 years old with a body mass index of 46.3 and had a history of hospital admissions due to bacterial pneumonias in the previous 3 years. Thirty-six patients (44.4%) received the complete treatment course of AZB comprising 10 injections, 24 patients (29.6%) received nine injections, 17 patients (21.0%) received eight injections, two patients (2.5%) received seven injections, and two patients (2.5%) each received six or two injections, respectively.

The results from this open-label, multi-center study in patients hospitalized with COVID-19 demonstrate that administration of AZB over a 17 day treatment period was associated with an improvement in clinical status as assessed by OS and NEWS values. The main indicators of pneumonia and lung function (oxygen saturation (SpO₂), signs of pneumonia, body temperature, CRP) also showed gradual recovery and normalization. The fact that CRP did not increase indicates the lack of cytokine storm risk in patients treated with AZB. Clinical improvements observed in older patients (≥65 years of age) were similar to those observed in younger patients (<65 years), with similar values obtained by the end of study treatment. AZB was safe and well-tolerated, with no AEs considered to be related to study treatment. No apparent interactions between AZB and antivirals, anticoagulants, or hydroxychloroquine were noted, further confirming the safety of AZB.

No deaths occurred during the study period. However, as mentioned above, one patient died from complications related to an SAE of Klebsiella sepsis after study completion. Results from a retrospective Russian study of 1522 patients with SARS-CoV-2 infection between March and May 2020 identified mortality rates of 36.8% and 76.5% for patients who required noninvasive or invasive ventilation, respectively. It was therefore reasonable to expect higher mortality in the selected patient population given that 22.2% and 21.0% of patients required noninvasive and invasive ventilation, respectively. This finding is even more apparent when considering the relatively high incidence of patients exhibiting comorbidities and the number of older patients (≥65 years), since comorbidities and age are both known risk factors for severe disease progression and mortality.

In a recent study, treatment with the corticosteroid dexamethasone reduced Day 28 mortality only in patients requiring mechanical ventilation or oxygen alone, with no significant benefit observed in patients not requiring respiratory support. In the present study, Day 28 mortality was not observed in any subgroup of patients who were treated with AZB, suggesting a treatment advantage of AZB over dexamethasone, particularly in patients with non-severe COVID-19. Furthermore, corticosteroid use has been associated with increased mortality in other respiratory illnesses, such as the flu, which could be due to the known association between corticosteroids and immunosuppression. With known immunomodulating properties, AZB could be used in place of or in conjunction with corticosteroids in patients requiring mechanical ventilation to balance the deleterious effects on the immune system and improve overall recovery. These observations merit further investigation.

As mentioned above, improvements in both OS and NEWS values from baseline to Day 17 were observed in the present study. However, there were notable differences in the behavior of the two parameters. The mean OS values were initially relatively stable during the first seven days of treatment followed by a notable decrease. The decrease in mean NEWS values was more pronounced, with improvements noted several days earlier, suggesting that OS score is less responsive to early changes in clinical improvement. This leads to the conclusion that NEWS may provide a more sensitive interpretation of patient clinical status and better reflect overall recovery as compared to the OS score.

The present study was an open-label study, the primary limitation of which was the absence of a formal control group. However, as pointed out above, historical control data was therefore identified (from Cao et al., supra, 2020) and included to provide a comparable group of patients who did not receive active treatment. The two groups (i.e., the historical control group and the AZB treatment group) had comparable demographics, baseline respiratory support requirements and duration of hospitalization. In the historical control group, however, a higher mortality rate was observed, and a lower proportion of patients exhibited clinical recovery (per OS scores) as compared to the group of patients treated with AZB in the present study.

Key differences between the AZB and the historical control studies were apparent, including that the historical control study described in Cao et al. was conducted several months earlier in China. Notably, countries have been affected by COVID-19 at different times and at different rates, so it is difficult to compare data when treatment options and experience may have been varied. Furthermore, SOC recommendations in China (used in the historical control data taken from Cao et al.) differed from the SOC recommendations of the Russian Federation (used in the present study). As such, the specific treatment benefit that can be attributed solely to AZB between the AZB treatment group and the historical control group is difficult to quantify. A comparison of the data suggests that some clinical benefit may have been achieved in patients administered AZB, whether directly related to the drug or otherwise. To address this, a randomized, double-blind, placebo-controlled clinical trial is currently underway.

Based on the previous studies of AZB, there may be several explanations for the nature of the observed effect (i.e., alleviation of symptoms and reduction of sick days) in patients with COVID-19. First, AZB can cause indirect antiviral activity by stimulating interferon release, antigen presentation and antibody development, facilitating the immune anti-COVID-19 response. Second, the detoxicant effect of AZB can participate in symptom reduction (e.g., temperature) and increased well-being in some patients. Another possible explanation of AZB effect is the prevention of cytokine storm, which serves as a predictive marker of poor COVID-19 outcome.

### EXAMPLE 2

From July 1 to Nov. 30, 2020, an open non-randomized study was conducted to assess the effectiveness and safety of the prophylactic use of AZB in medical workers who are in regular contact with patients infected with COVID-19. The study included 913 medical staff personnel aged from 24 to 59 years and working in the “red” zones of several hospitals in Russia.

The study assessed the number of acute respiratory infections and COVID-19 infections within 2 months (i.e., for the period of administering a prophylaxis of AZB for 1 month, and for 1 month after the last dose of AZB prophylaxis was administered) by interviewing medical personnel using specially prepared questionnaires. Then, to assess the duration of the effect of AZB as an anti-COVID-19 prophylactic, the second stage of the survey was carried out, in which the number of cases of acute respiratory infection and COVID-19 was estimated during the period of administering AZB for 30 days and within 3 months after administering the last dose of the AZB prophylaxis. 350 medical workers (of the 913 who participated in the first stage of the survey) took part in the second stage of the survey. Of these study participants, 247 people were administered AZB and 103 people were not administered any medications.

Inclusion criteria: a medical worker, by the nature of his/her professional activity, forced to contact patients, conduct examination, perform care and medical manipulations in patients infected by COVID -19, including cleaning the premises where patients infected by COVID-19. Exclusion criteria: a medical worker who has recovered or tested positive for COVID -19; unwillingness to participate in research and comply with protocol procedures; history of hypersensitivity to the study drug; history of acute or chronic renal failure; pregnancy or breastfeeding; rare hereditary lactose intolerance, lactase deficiency; a history of glucose-galactose malabsorption syndrome; participation as a subject in any other clinical study during the course of this study, including participation in a study within 30 days prior to the commencement of that study; and receiving cytostatic therapy or other drugs with an immunosuppressive effect, immunomodulators or immunostimulants within 60 days before the start of the study.

All study participants were divided into 2 groups: the main group (n=577) included medical workers received AZB (Polyoxidonium®) tablets 12 mg (under the tongue) once a day for 30 days, and the control group (n=336) included medical workers who did not receive any prophylactic drugs. The statistical analysis of the survey results was carried out using the STATA v.14 software (StataCorp, USA). Descriptive statistics were used in the analysis for quantitative variables, the mean value (Mean), standard deviation (SD), 95% confidence interval for the mean (95% CI), minimum value (Min), maximum value (Max), median (Me) are presented, first and third quartiles (Q1 and Q3); for qualitative variables, the absolute number in each category and the percentage (%) are presented. When comparing groups by numerical variables, the Student's t-test (unpaired) was used, while the hypothesis was tested for normal distribution (using the Kolmogorov-Smirnov test). When comparing groups for qualitative variables, the chi-square test (or Fisher's exact test, where applicable) was used. The level of statistical significance is accepted as p<0.05. The first stage of the survey was conducted with the participation of 913 medical workers, and the overall results are shown in Table 4.

TABLE 4
Distribution of participants by sex, age, presence of chronic diseases

| | Main (AZB) | | Control (Comparative) | |
|---|---|---|---|---|
| Evaluated parameters | Group | | Group | |
| Number of Participants Evaluated | n = 576* | | n = 335* | |
| Men | 266 | 46.2% | 167 | 49.9% |
| Women | 310 | 53.8% | 168 | 50.1% |
| p-value (chi-square test) | 0.2848 | | | |
| Number of Participants Evaluated | 577 | | 335** | |
| Mean age (Mean) ± standard deviation (SD), years | 41.63 ± 6.66 | | 41.56 ± 6.40 | |
| p-value (unpaired t-test) | 0.8768 | | | |
| Number of Participants Evaluated | 577 | | 336 | |
| Number of participants with chronic diseases | 312 | 54.1% | 121 | 36.0% |
| Number of participants without chronic diseases | 265 | 45.9% | 215 | 64.0% |

| | |
|---|---|
| p-value (chi-square test) | <0.0001 |

*2 participants-gender not specified
**1 participant-age not specified

Table 4 shows that the study groups are comparable in terms of gender and age (p>0.05). In the main group, the proportion of medical workers suffering from chronic diseases is statistically significantly higher (p<0.0001). Among the chronic diseases in both groups, the most common chronic infectious and inflammatory diseases of the respiratory system, such as chronic tonsillitis (140 people), chronic bronchitis (83 people), chronic sinusitis (51 people), COPD (47 people), chronic frontal sinusitis (11 people). Also, 50 people indicated obesity as a chronic disease, 32 people indicated gastric ulcer and 19 people indicated arterial hypertension as a chronic disease. In both groups, a comparable proportion of participants was vaccinated against influenza in 2019 (73.4% in the main group and 70.8% in the control group, p=0.3956). The number of people vaccinated against pneumococcus over the past 5 years in both groups was insignificant (6.8% in the main group and 3.0% in the control group).

In 2019, in the main group, medical workers had an acute respiratory infection significantly more often than participants in the control group (the average number of acute respiratory infection (ARI) cases was 1.34 (SD: 0.47) in the main group and 1.24 (SD: 0.43) in control group, p=0.0018). Also, for the period from Mar. 15, 2020 to Jul. 15, 2020, in the main group (before the start of taking the AZB prophylaxis), there was a significantly higher number of acute respiratory (ARI) cases compared to the control group, namely, 78 (13.5%) in the main group, and 29 (8.6%) in the control group, p=0.0262). It is likely that the greater number of ARI cases in 2019 and from March to July of 2020 in the main group is associated with a large number of chronic infectious and inflammatory diseases of the respiratory system in the group members.

TABLE 5
Number of cases of ARI and COVID-19 during the period of observation
(Jul. 15, 2020 to Sep. 15, 2020)

| | Main | | Control | |
|---|---|---|---|---|
| | (AZB) | | (Comparative) | |
| Evaluated parameters | Group | | Group | |
| Number of Participants Evaluated | 577 | | 336 | |
| Afflicted by acute respiratory illness (ARI) | 4 | 0.7% | 41 | 12.2% |
| p-value* | <0.0001 | | | |
| Of all acute respiratory illness cases, confirmed diagnosis of | 2 | 0.3% | 17 | 5.1% |
| COVID-19 (based on PCR or antibody test) | | | | |
| p-value** | <0.0001 | | | |
| Number of participants who developed pneumonia (reported | 0 | 0% | 17 | 42.5% |
| acute respiratory illness cases) | | | | |

*Chi-square test
**Fisher's exact test

Table 5 shows the number of cases of ARI and COVID-19 during the time of taking AZB in the main group and for the same period of time in the control group. Thus, in the control group, the absolute number of ARI cases is more than 10 times higher than in the main group (which received AZB prophylaxis). Also, in the control group, in 41.4% of ARI cases, according to testing, the SARS-CoV2 virus was detected, or antibodies to it were subsequently detected.

In the control group, 17 participants suffered pneumonia, and according to the PCR test or antibody test, pneumonia was caused by a new coronavirus infection in 15 medical workers (88.2%) of the control group. Although two cases of new coronavirus infection were also noted in the main group, they proceeded in the form of a mild upper respiratory tract infection, there were no cases of pneumonia. Since there were significantly more participants with chronic diseases in the main group, an additional assessment was conducted of the number of cases of ARI and COVID-19 in healthcare workers with chronic diseases. Table 5 also shows the number of participants with chronic conditions. Subgroups of participants isolated from the control group and the main group and suffering from chronic diseases are comparable in terms of distribution by sex, age, number of vaccinated against influenza and pneumococcus, and the number of ARI cases in the previous year and 4 months prior to the start of the study (p>0.05).

TABLE 6
Number of cases of ARI and COVID-19 in medical workers having chronic
illnesses during the period of observation (Jul. 15, 2020 to Sep. 15, 2020)

| | Main | | Control | |
|---|---|---|---|---|
| | (AZB) | | (Comparative) | |
| Evaluated parameters | Group | | Group | |
| Number of Participants Evaluated | 312 | | 121 | |
| Afflicted by acute respiratory illness (ARI) | 1 | 0.3% | 34 | 28.1% |
| p-value* | <0.00001 | | | |
| Of all acute respiratory illness cases, confirmed diagnosis of | 1 | 0.3% | 12 | 9.9% |
| COVID-19 (based on PCR or antibody test) | | | | |
| p-value* | <0.0001 | | | |
| Number of participants who developed pneumonia (reported | 0 | 0% | 12 | 35.3% |
| acute respiratory illness cases) | | | | |

*Chi-square test

As can be seen in Table 6, the number of ARI cases in the control group (34) significantly (more than 30 times) exceeds the number of cases in the main group (1). In the control group, the SARS-CoV2 virus was detected in 35.3% of ARI cases, according to testing data. Twelve participants in the control group suffered pneumonia, while according to the PCR test or antibody test, pneumonia was caused by a new coronavirus infection in ten medical workers (83.3%). In the main group, one case of COVID-19 was noted (according to the antibody test), and the disease was not accompanied by pneumonia.

When assessing the number of ARI cases in participants of the two groups who did not suffer from chronic diseases, a statistically significant difference between the number of ARI and COVID-19 cases in the main group and the control group as not seen (p=0.12011). This result is most likely explained by the relatively small number of ARI cases during the observed period in both groups (3 cases of ARI, of which 1 case of COVID-19 in the main group and 7 cases of ARI, of which 5 cases of COVID-19 in the control group). In addition, the period of observation was from July 15 to September 15, and these months are not typically the months historically associated with the epidemic rise of ARI. Accordingly, in these months, persons without chronic diseases are less at risk of contracting respiratory infectious diseases than persons with chronic diseases (especially those with chronic diseases of the respiratory tract, which were the majority, both in the main group and in the control group).

Interestingly, the number of ARI and COVID-19 cases in the main group in subgroups with and without chronic diseases (1 case of COVID-19 in the group with chronic diseases and 3 cases of ARI, of which 1 case of COVID-19) is almost the same. On the other hand, in the control group the number of ARI cases in the subgroup of persons with chronic diseases is almost 5 times higher than in the subgroup without chronic diseases (34 cases of ARI versus 7 cases of ARI, respectively). Thus, it can be assumed that the use of AZB as a prophylaxis makes it possible to further reduce the risks of infection for people with chronic diseases that they have due to the presence of these diseases.

To assess the duration of the effect of taking AZB, a second stage of questioning the medical personnel included in the main group and the control group was carried out. Data from 350 questionnaires of study participants was received in this second stage.

TABLE 7
Distribution of participants in the second stage of the survey by gender, age,
presence of chronic diseases

| | Main (AZB) | | Control (Comparative) | |
|---|---|---|---|---|
| Evaluated parameters | Group | | Group | |
| Number of Participants Evaluated | n = 247 | | n = 103 | |
| Men | 119 | 48.2% | 50 | 48.5% |
| Women | 128 | 51.8% | 53 | 51.5% |
| p-value (chi-square test) | 0.95027 | | | |
| Average age ± standard deviation, years | 41.96 ± 6.76 | | 41.25 ± 7.22 | |
| p-value (unpaired t-test) | 0.39628 | | | |
| Estimated number of participants | 245* | | 103 | |
| Participants with Flu Shots in 2020 | 169 | 69.0% | 75 | 72.8% |

| | |
|---|---|
| p-value (chi-square test) | 0.47548 |

*2 participants-no data

As can be seen from Table 7, the main and control groups in the case of the second stage of the survey were also comparable in terms of distribution by sex, age and number of influenza vaccinations in 2020. The second round of the survey estimated the number of people vaccinated against influenza in 2020, since the survey was conducted in November 2020, and by that time all participants could be vaccinated. In the first stage of the survey, it was possible to estimate the number of people vaccinated in 2019 only, since at the time of the survey, the 2020 vaccination period had not yet started. Also, the number of people vaccinated against COVID-19 was not estimated, since at the time of the surveys, a widespread COVID-19 vaccination campaign had not yet started in the geographic regions in which the study was conducted. At the same time, the proportion of those vaccinated against influenza in 2020 did not significantly differ from the proportion of those vaccinated in 2019, which suggests that the adherence to influenza vaccination among medical workers did not increase against the background of the coronavirus pandemic.

TABLE 8
The number of cases of ARI and COVID-19
for 4 months (of which the first month is the
month of administering AZB to the main group)

| | | | Control | |
|---|---|---|---|---|
| | Main (AZB) | | (Comparative) | |
| | Group | | Group | |
| | n = 245* | | n = 103 | |
| Afflicted by ARI | 32 | 13.1% | 59 | 57.3% |
| p-value (chi-square test) | <0.000001 | | | |
| Of all ARI cases, a confirmed | 6 | 2.4% | 27 | 26.2% |
| diagnosis of COVID-19 | | | | |
| (based on PCR test data) | | | | |

| | |
|---|---|
| p-value (Fisher's exact test) | <0.01206 |

*2 participants-no data

It can be seen in Table 8 that the number of ARI cases and the number of COVID-19 cases are statistically significantly lower in the main group, which may indicate that the preventive effect of AZB persists for some time even after the end of its administration. 86.9% of the participants in the main group indicated in the survey that they considered the prophylaxis with AZB to be effective, and 88.2% of the participants indicated in the survey that they would like to continue prophylaxis with AZB in the future.

According to the data presented above in Tables 4-8, the number of medical workers with chronic diseases in the main (AZB Prophylaxis) group is higher than in the control (No Prophylaxis) group. This discrepancy is probably explained by the fact that study participants with chronic diseases, realizing the additional risks for them, are more inclined to use additional preventive measures. Also, in both groups, there was a high level of influenza vaccinations in 2019 and in 2020 (about 70% and more). This level was generally higher than the Russian national average (50.5% of the population were vaccinated against influenza in 2019 according to Rospotrebnadzor data) and higher than the average (36.7%) among doctors of various medical specialties.

Interestingly, among medical students, only 23.1% of junior students are vaccinated against influenza, while 50% of the senior students are vaccinated. Vaccination rates against pneumococcus continue to be low in Russia. In the present study, the proportion of those vaccinated against pneumococcal infection was less than 10% for both groups. In general, in 2019 and for 4 months of 2020, medical workers from the main group were sick more often than participants in the control group, which is probably due to the fact that the proportion of participants with chronic infectious and inflammatory diseases is higher in the main group.

Based on the data obtain in the study of prophylactic administration of AZB to medical workers, the following conclusions can be drawn, which are discussed below.

First, in the group that was administered AZB as a prophylactic treatment, a significant decrease in the number of cases of ARI and COVID-19 was recorded in comparison with the control group. In particular, according to the data of the first stage of the survey, 0.7% of participants in the main group fell ill with ARI, 0.3% of COVID-19. In the control group, 12.2% fell ill with ARI, 9.9%-COVID-19. According to the data of the second stage of the survey, 13.1% of participants in the main group fell ill with ARI, 2.4% of COVID-19. In the control group, 57.3% fell ill with ARI and 26.2% with COVID-19.

Second, the present study strongly suggests that a prophylactic treatment of people with AZB as described herein likely contributes to a milder course of COVID-19 (i.e., less severe symptoms, shorter duration of sickness), should the person who was administered the prophylaxis still get infected with COVID-10. In the main group, none of the participants contracted pneumonia, while in the control group, pneumonia was noted in 42.5% of ARI cases.

Third, a prophylaxis with AZB significantly reduces the number of cases of ARI and COVID-19 among healthcare workers suffering from chronic diseases. Notably, with AZB prophylaxis, the number of ARI cases in participants with chronic diseases is comparable to the number of ARI cases without chronic diseases. It is possible that the prophylactic treatment with AZB reduces the additional risks of infection in patients with chronic diseases due to the presence of these diseases.

Fourth, 86.9% of the surveyed medical workers considered AZB to be effective in preventing ARI and COVID-19 and 88.2% of the surveyed medical workers indicated that they would like to again receive AZB prophylaxis in the future.

Overall, this study shows that a prophylactic administration of AZB leads to a decrease in the incidence of ARI and COVID-19 in medical workers, both during the period of using the drug and within three months after taking it. Without wishing to be limited by theory, prophylactic administration of AZB is thought to increase the activity of lysozyme and the production of secretory immunoglobulin A, and can help maintain these parameters at a high level for about 3 months after the end of the AZB prophylactic treatment. Notably, the surveyed medical workers indicated that the proposed dosage regimen is convenient (once a day under the tongue), which contributes to the high adherence of medical workers to this method of prevention. Thus, the use of AZB as a prophylaxis to prevent ARI and COVID-19 meets modern requirements and can serve as an additional line of defense against ARI and COVID-19 infection not only for medical workers, but also for patients, especially those with chronic diseases.

The devastating impacts of the COVID-19 pandemic are far from over and global collaboration to find effective treatments is paramount. The recent deployment of approved vaccines in several countries represents a huge milestone. However, continuing research into other treatments is vital. The promising safety and efficacy results described herein indicate that, in appropriately controlled conditions, AZB could serve well both as a treatment for patients with COVID-19, and as a prophylactic measure that could prevent and/or reduce the severity of COVID-19 infection in people who are exposed to COVID-19.

Those skilled in the art will recognize that a wide variety of other modifications, alterations, and combinations can also be made with respect to the above described embodiments without departing from the scope of the invention, and that such modifications, alterations, and combinations are to be viewed as being within the ambit of the inventive concept.

What is claimed is:

1. A method of treating COVID-19, the method comprising administering to a subject, who is infected with COVID-19, a therapeutically effective amount of azoximer bromide.

2. The method of claim 1, wherein the administering the therapeutically effective amount of the azoximer bromide comprises injecting 12 mg of azoximer bromide intravenously once per day on days 1, 2, and 3 of treatment, and then intramuscularly on day 5 and every other day following day 5 until day 17, for a total of 10 injections.

3. The method of claim 1, wherein the administering of the therapeutically effective amount of the azoximer bromide comprises injecting 6 mg of azoximer bromide intramuscularly once per day on days 1, 2, and 3 of treatment, and then every other day following day 5 until day 17, for a total of 10 injections.

4. The method of claim 1, wherein the administering of the therapeutically effective amount of the azoximer bromide comprises administering one 12 mg tablet of azoximer bromide twice per day for 7 consecutive days.

5. The method of claim 1, wherein the administering of the therapeutically effective amount of the azoximer bromide comprises spraying 6 mg of azoximer bromide intranasally three times per day for 10 consecutive days.

6. The method of claim 6, wherein the administering of the therapeutically effective amount of the azoximer bromide comprises administering a 12 mg suppository of azoximer bromide once per day for 10 consecutive days.

7. The method of claim 1, further comprising administering the therapeutically effective amount of azoximer bromide in combination with at least one of a vitamin, a mineral, an antibiotic, an antiviral, an immunosuppressant, an hydroxychloroquine, and an anti-coagulant.

8. A method of prophylaxis against infection with COVID-19, the method comprising administering to a healthy subject who is not infected with COVID-19, a therapeutically effective amount of azoximer bromide.

9. The method of claim 8, wherein the administering of the therapeutically effective amount of the azoximer bromide comprises injecting 12 mg of azoximer bromide intravenously once per day on days 1, 2, and 3 of prophylaxis, and then intramuscularly on day 5, and then on every other day following day 5 until day 17, for a total of 10 injections.

10. The method of claim 8, wherein the administering of the therapeutically effective amount of the azoximer bromide comprises injecting 6 mg of azoximer bromide intramuscularly once per day on days 1, 2, and 3 of prophylaxis, and then on day 5 and every other day following day 5 until day 17, for a total of 10 injections.

11. The method of claim 8, wherein the administering of the therapeutically effective amount of the azoximer bromide comprises administering one 12 mg tablet of azoximer bromide once per day for at least 10 consecutive days.

12. The method of claim 11, wherein the administering of the therapeutically effective amount of the azoximer bromide comprises administering one 12 mg tablet of azoximer bromide once per day for at least 20 consecutive days.

13. The method of claim 12, wherein the administering of the therapeutically effective amount of the azoximer bromide comprises administering one 12 mg tablet of azoximer bromide once per day for 30 consecutive days.

14. The method of claim 8, wherein the administering of the therapeutically effective amount of the azoximer bromide comprises spraying 6 mg of azoximer bromide intranasally three times per day for 10 consecutive days.

15. The method of claim 8, wherein the administering of the therapeutically effective amount of the azoximer bromide comprises administering a 12 mg suppository of azoximer bromide at least once per day for 10 consecutive days.

15. The method of claim 8, wherein the administering of the therapeutically effective amount of the azoximer bromide comprises administering a 12 mg suppository of azoximer bromide twice per day for 10 consecutive days.
